(19) United States
(12) Patent Application Publication
(10) Pub. No.: US 2020/0030399 A1
(43) Pub. Date: Jan. 30, 2020

(54) HERBAL COMPOSITIONS OF CALIFORNIA POPPY AND METHOD FOR THE TREATMENT OF SLEEP AND DREAM-RELATED DISORDERS

(71) Applicant: MONDIAS NATURELS INC., Longueuil, Quebec (CA)

(72) Inventor: Guy Chamberland, Boucherville, Quebec (CA)

(21) Appl. No.: 16/485,634

(22) PCT Filed: Feb. 13, 2018
(86) PCT No.: PCT/CA2018/050161

§ 371 Date: Aug. 13, 2019

Related U.S. Application Data
(60) Provisional application No. 62/458,230, filed on Feb. 13, 2017.

(51) Int. Cl. A61K 36/66; A61K 31/47; A61P 25/20; A61P 25/24; A61P 25/04; A61P 25/18

(52) U.S. Cl. CPC A61K 36/66; A61K 31/47; A61P 25/18; A61P 25/24; A61P 25/04; A61P 25/20

(57) ABSTRACT

An herbal pharmaceutical compositions of California poppy and methods for the treatment of sleep disorders and dream-related disorders is disclosed. The formulation may be in the form of capsules, tablets, caplets or other oral dosage forms and comprises preferably 600 to 2400 mg of a ratio of 4-10 parts (wt) dried plant material to 1 part (wt) aqueous alcohol (50-90 vol % ethanol) extract that is equivalent to 3-12 grams of the dried herb top of California poppy and contain at least 0.8 wt % isoquinoline alkaloids. The composition may further comprise isoquinoline alkaloids whose composition contains Californidine, Escholtzine and Protopine. California poppy can also be used as a therapy for managing people that have sleep and dream disorders such as Post Traumatic Stress syndrome and similar conditions that induce nightmares as well as people using tranquilizer, antidepressant, antipsychotic, sedative, hypnotic and analgesic drugs.

### CROSS-REFERENCE TO RELATED APPLICATIONS

The present patent application claims the benefits of priority of commonly assigned U.S. Provisional Patent Application No. 62/458,230, entitled “HERBAL COMPOSITIONS OF CALIFORNIA POPPY AND METHOD FOR THE TREATMENT OF SLEEP AND DREAM-RELATED DISORDERS” and filed at the United States Patent and Trademark Office on Feb. 13, 2018, the content of which is incorporated herein by reference.

### FIELD OF THE INVENTION

The present invention generally relates to herbal compositions of California poppy and methods for the treatment of sleep disorders and dream-related disorders.

### BACKGROUND OF THE INVENTION

Historical medical references considered remedies such as Opium Poppy of great use as an anodyne and analgesic. Felter and Lloyd (1983) considered Eschscholzia californica to be an “ . . . analgesic and soporific without the dangers attending opiates, quieting pain and producing a calm sleep”. California poppy (Eschscholzia californica Cham. (Ec)) is an annual herb that has been traditionally used for its analgesic, anxiolytic and sedative (hypnotic) properties (Meolie et al., 2005; van Wyk et Wink 2010). Both the root and leaves had therapeutic uses as analgesics and sedatives in Native American medicine (Moerman 1998) and in Eclectic medicine in the late 1800s and early 1900s (Felter et Lloyd 1983).

The Spaniards were the first to discover the therapeutic use of this herb by Natives in California. It was during a Russian expedition that the herb was named after the ship's surgeon and naturalist (i.e., Eschscholz). The expedition brought specimens back to Europe where it subsequently became part of the botanical medicine practice (Holmes 2006). California poppy has been used by Native Americans and Eclectic physicians as a sedative, analgesic and anxiolytic (Felter and Lloyd 1983; Sarris 2007).

In phyto-energetic medicine, California poppy is recognized as an analgesic, sedative and neuromuscular relaxant. The herb is used to treat insomnia, pain and anxiety by reducing floating Yang and activating constrained Qi (Holmes 2006). Its traditional use is described in monographs of the German E Commission (Blumenthal 2000) and Health Canada. When used as a sleep aid, the time it takes to fall asleep is reduced and sleep quality is purported by users as improved (van Wyk et Wink 2010).

Based on common knowledge of pharmacology and herbal science, use of California poppy in combination with tranquilizers, sedatives, analgesics, antidepressants, hypnotics, and some antipsychotics is expected to result in an additive anxiolytic, analgesic, sedative or hypnotic effect.

A 5:1 (5 parts herb top to 1 part solvent) herb extract has also been shown to possess anxiolytic, sedative and analgesic properties. The pharmacological mechanism of action of California poppy has been investigated. Dose-dependent sedative properties as well as anxiolytic effects were shown in mice (Rolland et al., 1991). The anxiolytic effects occurred at lower doses than the sedative effects. In a subsequent study, using a 5:1 dried herb extract (aqueous alcohol 60%¹), Rolland et al (2001) confirmed the sedative and anxiolytic properties of the herb in mice using two behavioural tests. Flumazenil® is a benzodiazepine antagonist and was used to antagonize (partial antagonism) the sedative and anxiolytic effects. The mouse study also demonstrated that the herb had no antihistaminic effects which have been associated with sedative effects of some drugs. This led the researchers to suggest that benzodiazepine receptors may be implicated in the sedative and anxiolytic properties of the herb. Contrary to the typical pharmacological properties of a benzodiazepine, California poppy did not exhibit any anticonvulsant properties against pentetylenetetrazole (Rolland et al., 2001). Similarly, the mouse study also showed that the herb lacked muscle relaxant and antipsychotic properties which are common with benzodiazepines and antidepressants. The herb also lacked antidepressant-like properties in the animal model. A dose-dependent peripheral analgesic effect was demonstrated using the writhing test. At higher dose levels, the herb had slight benefits in the hot plate. These results suggest that the herb has both a peripheral analgesic effect and a weaker central analgesic action. ¹ extract is a 5:1¹ aqueous alcohol (70%) extract.

Gafner et al (2006) demonstrated that a 1:10² aqueous alcohol (70%) extract of California poppy was able to bind to 5-HT1A and 5-HT7 receptors. The activity on the 5-HT(1A) receptor was at least partly due to the presence of the aporphine alkaloid 3 (Gafner et al., 2006). The dose-dependent antidepressant-like effects of protopine were demonstrated in animal models (Xu et al., 2006). Protopine was shown to be an inhibitor of both serotonin transporter and noradrenaline transporter in vitro. ² A 1:10 extract ratio=1 part of dried herb to 10 parts solvent.

The following is a summary of the animal pharmacology (mice) performed using a 5:1 aqueous alcohol (60%) extract of California poppy.
The sedative effects of the extract were partly antagonized by Flumazenil (benzodiazepine receptor antagonist). The extract produced similar effects to Midazolam (Versed®).
The anxiolytic effects of the extract were antagonized by Flumazenil (benzodiazepine receptor antagonist). The effects were similar to those obtained with Clorazepate dipotassium.
No anticonvulsant or myorelaxant effects were observed. Dose-dependent anticonvulsant and myorelaxant effects were seen with Clorazepate in this study.
No protective effect was observed against dexamphetamine produced mortality. Whereas, a protective effect was observed with Chlorpromazine.
A dose-response was observed. The extract significantly increased the stereotyped behaviour effects of dexamphetamine whereas Chlorpromazine significantly decreased these effects.
The extract had no effect against reserpine-induced ptosis, akinesia or hypothermia. Whereas, amitryptiline significantly reduced ptosis and hypothermia.
The extract had no effect against the anticholinergic effects of Oxotremorine. Whereas, Imipramine significantly reduced these effects.
A dose-response effect was observed for peripheral analgesia against acetic acid writhing. Protection against writing was 70% and 85% with the extract whereas acetylsalicylic acid, paracetamol and morphine sulphate gave protection at 60%, 60% and 70% respectively.

California poppy has been shown to contain the following alkaloids: californidine, escholtzine, protopine, N-methyllaurotetanine, caryachine, O-methylcaryachine, and the pavine alkaloid, 6S,12S-neocaryachine-7-O-methyl ether N-metho salt (Gafner et al. 2006). The main alkaloids of the leaves are the isoquinoline alkaloids californidine, escholtzine and protopine which are associated with its known properties associated with the prior art therapeutic properties.

### SUMMARY OF THE INVENTION

The shortcomings of the prior art are generally mitigated by Herbal compositions of California poppy (Eschscholzia californica Cham.) for the treatment of sleep and dream-related disorders.

It is also disclosed the use of the herbal pharmaceutical composition as defined in herein, for the treatment of sleep disorders and/or dream-related disorders.

It is also disclosed the use of California poppy for the making of an herbal pharmaceutical composition for the treatment of sleep disorders and/or dream-related disorders.

It is also disclosed a method for treating sleep disorders and/or dream-related disorders, the method comprising the step of administering the pharmaceutical composition as defined herein.

The California poppy formulations will improve nonrestorative sleep in patients by a pharmacological effect on sleep patterns that is not associated with the herb's analgesic, anxiolytic, sedative or hypnotic effects.

In one aspect of the invention, an herbal pharmaceutical composition of California poppy (Eschscholzia californica Cham.) for the treatment of sleep disorders and/or dream-related disorders is provided. The composition comprises an extract of dried herb top of California poppy having a ratio of 5 parts (wt) of dried plant material to 1 part (wt) of aqueous alcohol (50-90 vol % ethanol). The extract may comprise between 3 grams to 12 grams of dried plant material of California poppy and/or the composition may further comprise 600 mg of the extract of dried herb top of California poppy.

The composition may comprise at least 0.8 wt %, but not more than 1 wt %, isoquinoline alkaloids, the isoquinoline alkaloids comprising Californidine (60.0-80.0 wt % total alkaloids), Escholtzine (10.0-30.0 wt % total alkaloids) and Protopine (3.0-10.0 wt % total alkaloids).

In another aspect of the invention, an herbal pharmaceutical composition of California poppy (Eschscholzia californica Cham.) for the treatment of sleep disorders and/or dream-related disorders is provided. The composition comprised at least between 600 to 2400 mg of an extract of California poppy having a ratio of between 4 to 10 parts (wt) dried plant material to 1 part (wt) aqueous alcohol (50-90 vol % ethanol). The extract may comprise between 3 grams to 12 grams of dried plant material of California poppy and/or the composition may comprise at least 0.8 wt % isoquinoline alkaloids, the isoquinoline alkaloids comprising Californidine (60.0-80.0 wt % total alkaloids), Escholtzine (10.0-30.0 wt % total alkaloids); and Protopine (3.0-10.0 wt % total alkaloids).

The composition may be in the form of capsules, tablets, caplets or other oral dosage forms.

In a further aspect of the invention, the use of herbal pharmaceutical composition of California poppy (Eschscholzia californica Cham.) comprising an extract of dried herb top of California poppy having a ratio of 5 parts (wt) of dried plant material to 1 part (wt) of aqueous alcohol (50-90 vol % ethanol) for the treatment of sleep disorders and/or dream-related disorders is provided.

The composition may be used for sleep disorders and/or dream-related disorders associated with Post Traumatic Stress syndrome and similar conditions that induce nightmares.

The composition may be used for sleep disorders and/or dream-related disorders associated associated with the use of tranquilizer, antidepressant, antipsychotic, sedative, hypnotic and analgesic drugs.

In another aspect of the invention, the use of California poppy (Eschscholzia californica Cham.) for the treatment of treatment for sleep disorders and/or dream-related disorders is provided.

In a further aspect of the invention, a method for treating sleep disorders and/or dream-related disorders is provided. The method comprises administering a pharmaceutical composition of California poppy (Eschscholzia californica Cham), the composition being in the form of a capsule. The method may further comprise given 2 capsules of the formulation before going to sleep.

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### DETAILED DESCRIPTION OF THE PREFERRED EMBODIMENT

A novel pharmaceutical composition will be described hereinafter. Although the invention is described in terms of specific illustrative embodiments, it is to be understood that the embodiments described herein are by way of example only and that the scope of the invention is not intended to be limited thereby.

It is disclosed herbal compositions of California poppy for the treatment of sleep and dream-related disorders.

According to a preferred embodiment, the formulation is in a form of Capsules, tablets, caplets or other oral dosage forms that preferably contain and or deliver 600 to 2400 mg of a ratio of 4-10 parts (wt) dried plant material to 1 part (wt) aqueous alcohol (50-90 vol % ethanol) extract that is equivalent to 3-12 grams of the dried herb top of California poppy and contain at least 0.8 wt % isoquinoline alkaloids (based on HPLC analysis). The composition of the isoquinoline alkaloids is:
Californidine (60.0-80.0 wt % total alkaloids by HPLC)
Escholtzine (10.0-30.0 wt % total alkaloids by HPLC)
Protopine (3.0-10.0 wt % total alkaloids by HPLC)

According to a preferred embodiment, the formulation is CALPXT96™ (formulation used in the ongoing trial; 2 capsules given at bedtime): Capsule that contains 600 mg of a ratio of 5 parts (wt) dried plant material to 1 part (wt) aqueous alcohol (50-90 vol % ethanol) extract that is equivalent to 3 grams of the dried herb top of California poppy and contains at least 0.8 wt %, but not more than 1 wt %, isoquinoline alkaloids (based on HPLC analysis). It has been discovered that these formulations alter the sleep pattern of the patient in such a way that the body will undergo a restorative sleep. This effect is characterized by changes in the dreaming of the patient; patient undergoes fantastic, pleasant or strange dreams.

Changes in the dreaming are associated with restorative sleep. The effect on the dreams is not caused by delusions or hallucinations induced by the herb formulation but by a pharmacological effect that acts on the sleep and dream patterns

According to the scientific/medical literature, 50% to 89% of chronic pain patients complain of poor sleep and/or feeling unrefreshed upon awakening (Okura et al. Sleep Medicine 2008; 9:352-361). Clinical studies have shown that pain interferes with sleep (Smith and Haythornthwaite. Sleep Med Rev 2004; 8:119-132) and that disturbed sleep lowers the pain threshold (Haack et al. Sleep 2007; 30:1145-1152; & Smith et al. Sleep 2007; 30:494-505; & Roehrs et al. Sleep 2006; 29:145-151.). It is also known that, in general, tranquilizers, antidepressants, antipsychotics, sedatives, hypnotics and analgesics decrease sleep in people. When California poppy is taken for sleep in people using tranquilizer, antidepressant, antipsychotic, sedative, hypnotic and analgesic drugs, the California poppy will still provide restorative sleep in these patients because it is not associated with drug's pharmacological mode of action; in other words, the restorative sleep is the not the result of an additive sedative, tranquilizing or hypnotic effect.

California poppy can be used as a therapy for managing people that have sleep and dream disorders such as Post Traumatic Stress syndrome and similar conditions that induce nightmares. The herb will act on the dream physiology to modify the imbalance and produce fantastic, pleasant or strange dreams that are not ‘scary’ or traumatizing to the person.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

### REFERENCES

Awad R et al (2007). Effects of traditionally used anxiolytic botanicals on enzymes of the g-aminobutyric acid (GABA) system. Can. J. Physiol. Pharmacol. 85: 933-942 (2007).
Blumenthal M, Goldberg A, Brinckmann J, editors. Herbal Medicine: Expanded Commission E Monographs. Boston (Mass.): Integrative Medicine Communications; 2000.
Chamberland G. Herbal remedies for pain management. Master Herbalist Thesis presented to the Dominion Herbal College of Canada, 2012.
Felter H W, Lloyd J U. King's American Dispensatory, Volume 2, 18th edition. Sandy (OR): Eclectic Medical Publications [Reprint of 1898 original] 1983.
Gafner S. et al. (2006). Alkaloids from Eschscholzia californica and their capacity to inhibit binding of [3H]8-Hydroxy-2-(di-N-propylamino)tetralin to 5-HT1A receptors in Vitro. J Nat Prod. 2006; 69(3):432-5.
Haack M. et al. (2007). Elevated Inflammatory Markers in Response to Prolonged Sleep Restriction Are Associated With Increased Pain Experience in Healthy Volunteers Sleep 2007; 30:1145-1152.
Hanus M, Jacqueline Lafon and Marc Mathieu (2004). Double-blind, randomised, placebo-controlled study to evaluate the efficacy and safety of a fixed combination containing two plant extracts (Crataegus oxyacantha and Eschscholtzia californica) and magnesium in mild-to-moderate anxiety disorders. Current Medical Research Opin 2004; 20(1):63-71.
Health Canada 2014. Monographs prepared by the Natural Health Products Directorate, Health Canada. http://webprod.hc-sc.gc.ca/nhpid-bdipsn/monosReq.do?lang=eng; Accessed 2014.
Holmes P. The Energetics of Western Herbs: A Materia Medica Integrating Western & Chinese Herbal Therapeutics. Snow Lotus Press, 2006.
Meolie, A. L., Rosen, C., Kristo, D., Kohrman, M., Gooneratne, N., Aguillard, R. N., Fayle, R., Troell, R., Townsend, D., Claman, D., Hoban, T., and Mahowald, M. Oral nonprescription treatment for insomnia: an evaluation of products with limited evidence. J Clin.Sleep Med 4-15-2005; 1 (2): 173-187.
Moerman D E. Native American Ethnobotany. Timber Press, 1998.
Okura K. et al (2008). Comparison of sleep variables between chronic widespread musculoskeletal pain, insomnia, periodic leg movements syndrome and control subjects in a clinical sleep medicine practice. Sleep Medicine 2008; 9:352-361.
Roehrs T. et al (2006). Sleep Loss and REM Sleep Loss are Hyperalgesic. Sleep 2006; 29:145-151.
Rolland A., Fleurentin J., Lanhers M C. et al (1991). Behavoral effects of the American traditional plant, Eschscholzia californica: sedative and anxiolytic properties. Planta Med 57:212-216 (1991).
Rolland A et al (2001). Neurophysiological Effects of an Extract of Eschscholzia californica Cham. (Papaveraceae). Phytother. Res. 15, 377-381 (2001).
Salminen K A et al (2011). Inhibition of human drug metabolizing cytochrome P450 enzymes by plant isoquinoline alkaloids. Phytomedicine 2011 Apr. 15; 18(6):533-8;
Sarris J. (2007). Herbal Medicines in the Treatment of Psychiatric Disorders: A Systematic Review. Phytother. Res. 21, 703-716 (2007).
Smith and Haythornthwaite (2004). How do sleep disturbance and chronic pain inter-relate? Insights from the longitudinal and cognitive-behavioral clinical trials literature. Sleep Medicine Reviews 2004; 8:119-132.
Smith M. et al (2007). The Effects of Sleep Deprivation on Pain Inhibition and Spontaneous Pain in Women. Sleep 2007; 30:494-505.
Treit D (1985). Animal models for the study of anti-anxiety agents: a review. Neuroscience and Biobehavioral Reviews 1985; 9:203-22.
van Wyk B E et Wink M, Medicinal Plants of the World, Timber Press, 2010.
White, R. E., (2000). High-throughput screening in drug metabolism and pharmacoki-netic support of drug discovery. Annu. Rev. Pharmacol. Toxicol. 40, 133-157.
Xu L F, et al (2006). Protopine inhibits serotonin transporter and noradrenaline transporter and has the antidepressant-like effect in mice models. Neuropharmacology 50 (2006) 934-940.

1. An herbal pharmaceutical composition of California poppy (Eschscholzia californica Cham.) for the treatment of sleep disorders and/or dream-related disorders, the composition comprising an extract of dried herb top of California poppy having a ratio of 5 parts (wt) of dried plant material to 1 part (wt) of aqueous alcohol (50-90 vol % ethanol).

2. The herbal pharmaceutical composition of California poppy of claim 1, the extract comprising between 3 grams to 12 grams of dried plant material of California poppy.

3. The herbal pharmaceutical composition of California poppy of any of claim 1 or 2, the composition comprising 600 mg of the extract of dried herb top of California poppy.

4. The herbal pharmaceutical composition of California poppy of any one of claims 1 to 3, the composition further comprising at least 0.8 wt %, but not more than 1 wt %, isoquinoline alkaloids, the isoquinoline alkaloids comprising:
Californidine (60.0-80.0 wt % total alkaloids);
Escholtzine (10.0-30.0 wt % total alkaloids); and
Protopine (3.0-10.0 wt % total alkaloids).

5. An herbal pharmaceutical composition of California poppy (Eschscholzia californica Cham.) for the treatment of sleep disorders and/or dream-related disorders, wherein said composition comprising at least between 600 to 2400 mg of an extract of California poppy having a ratio of between 4 to 10 parts (wt) dried plant material to 1 part (wt) aqueous alcohol (50-90 vol % ethanol).

6. The herbal pharmaceutical composition of California poppy of claim 5, the extract comprising between 3 grams to 12 grams of dried plant material of California poppy.

7. The herbal pharmaceutical composition of California poppy of any one of claim 5 or 6, the composition comprising at least 0.8 wt % isoquinoline alkaloids, the isoquinoline alkaloids comprising:
Californidine (60.0-80.0 wt % total alkaloids);
Escholtzine (10.0-30.0 wt % total alkaloids); and
Protopine (3.0-10.0 wt % total alkaloids).

8. The herbal pharmaceutical composition of California poppy of any of claims 1 to 7, wherein the composition is in the form of capsules, tablets, caplets or other oral dosage forms.

9. Use of herbal pharmaceutical composition of California poppy (Eschscholzia californica Cham.) of any one of claims 1 to 6 for the treatment of sleep disorders and/or dream-related disorders.

10. The use of the herbal pharmaceutical composition of claim 9, wherein the sleep disorders and/or dream-related disorders are associated with Post Traumatic Stress syndrome and similar conditions that induce nightmares.

11. The use of the herbal pharmaceutical composition of claim 9, wherein the sleep disorders and/or dream-related disorders are associated with the use of tranquilizer, antidepressant, antipsychotic, sedative, hypnotic and analgesic drugs.

12. Use of California poppy (Eschscholzia californica Cham.) for the treatment of treatment for sleep disorders and/or dream-related disorders.

13. The use of the herbal pharmaceutical composition of claim 12, wherein the sleep disorders and/or dream-related disorders are associated with Post Traumatic Stress syndrome and similar conditions that induce nightmares.

14. The use of the herbal pharmaceutical composition of claim 12, wherein the sleep disorders and/or dream-related disorders are associated with the use of tranquilizer, antidepressant, antipsychotic, sedative, hypnotic and analgesic drugs.

15. A method for treating sleep disorders and/or dream-related disorders, the method comprising the step of administering a pharmaceutical composition as defined in any one of claims 1 to 8, the composition being in the form of a capsule.

16. The method of claim 15, wherein 2 capsules of the formulation are given before going to sleep.

17. The method of any one of claim 15 or 16, wherein the sleep disorders and/or dream-related disorders are associated with Post Traumatic Stress syndrome and similar conditions that induce nightmares.

18. The method of any one of claim 15 or 16, wherein the sleep disorders and/or dream-related disorders are associated with the use of tranquilizer, antidepressant, antipsychotic, sedative, hypnotic and analgesic drugs.
